(19) United States
(12) Patent Application Publication
(10) Pub. No.: US 2025/0170087 A1
(43) Pub. Date: May 29, 2025

(54) AMPHOTERIC ALKYLAMIDO BETAINES PRODUCED FROM MACAÚBA PALM OIL

(71) Applicant: BASF SE, Ludwigshafen (DE)

(72) Inventors: Hendrik Huesken, Monheim (DE); Agustin Sanchez Valdivia, Monheim (DE); Allan Gustavo Picoli, San Paulo (BR)

(21) Appl. No.: 18/840,308

(22) PCT Filed: Feb. 23, 2023
(86) PCT No.: PCT/EP2023/054543

§ 371 Date: Aug. 21, 2024

(30) Foreign Application Priority Data
Feb. 25, 2022 (EP) 22158817.1
Feb. 25, 2022 (EP) 22158855.1
Feb. 25, 2022 (EP) 22158873.4
Feb. 25, 2022 (EP) 22158888.2

(51) Int. Cl. A61K 31/205; A61Q 5/02; A61Q 19/10

(52) U.S. Cl. CPC A61K 31/205; A61Q 5/02; A61Q 19/10; A61K 2800/805

(57) ABSTRACT

The present invention relates to a composition comprising alkylamido betaines derived from Macaúba palm oil.

### TECHNICAL FIELD

The present invention relates to amphoteric alklyamido betaine compositions derived from the oil of the fruits of the Macaúba palm.

### BACKGROUND

Numerous oil-based products are derived from renewable materials such as oil palm (principal source of palm oil). While such an approach is advantageous, since it safes the petroleum deposit, it also provides several downsides. One issue is the deforestation in order to plant e.g., oil palm plantations, which aggravates the current climate change. Deforestation further leads to undesired loss of biodiversity and the loss of habitats for local tribes. In addition, particularly oil palms need tropical conditions and preferred temperatures between about 24 to 28° C., monthly rainfalls of at least 100 mm/m², and a humidity between about 50 to 70%. These factors limit the possibility of a profitable cultivation.

At the same time the demand for renewable oil increases every year since the worldwide consume is increasing. Products derived from renewable oil can be found in every important industrial section, e.g., food products, pharmaceuticals, consumer goods, or energy (biodiesel).

Compounds classified as “amphoteric surfactants” are an important class of surfactants. Best known and widely used are the so-called alkylamido betaines, especially cocamido propyl betaines. These betaines are extensively used in personal care products, such as shampoos, shower gels and other cleansing products. They are considered as being very mild while having high foaming capacities.

### SUMMARY OF THE INVENTION

Nevertheless, alkylamido betaines derived from palm oil or palm kernel oil provide certain disadvantages. One property in question is the storage stability, which could be improved. As alkylamido betaines are base compounds for caring products, they are usually stored, shipped and further processed. It is therefore desirable to provide alkylamido betaines having improved storage stability, handling and processability properties.

Furthermore it is desirable to provide a personal care composition or a cleaning composition comprising alkylamido betaines having improved storage stability, application and performance properties.

Hence, there is an ongoing need for a more environmentally friendly alternative to alkylamido betaines derived from renewable oil such as palm oil or palm kernel oil. Furthermore, there is the need for alkylamido betaines derived from renewable oil resources, which show improved storage stability, handling and processability properties as well as improved storage stability, application and performance properties in personal care and cleaning products.

Hence, it is an object of the present invention to provide an alkylamido betaine composition having an improved sustainability profile and improved properties. Furthermore, it is an object of the present invention to provide an alkylamido betaine composition, wherein the starting material is derived from plants that are less vulnerable against temperature fluctuation. Furthermore, it is an object to provide an amphoteric surfactant having an improved sustainability profile and improved properties. Another object of the present invention is to provide an alkylamido betaine composition having improved storage stability and applicability for tailored care products. Finally, it is an object of the present invention to provide a personal care composition or a cleaning composition, having an improved sustainability profile.

In this connection, a more environmentally friendly alternative preferably provides at least one, more preferably at least two, still more preferably at least three, and in particular at least four of the following impacts: reduced water demand, reduction of the loss of biodiversity, reduction of loss of habitats for local tribes, reduction of deforestation, improved recovery of degraded areas and springs and watersheds, improved retention of moisture in the soil, improved resistance to temperature fluctuations and climate change.

It has surprisingly been found that at least one of these objects can be achieved by deriving the composition of alkyl amido betaines from an oil extracted from the Macaúba palm.

Thus, according to a first aspect, the present invention relates to a composition comprising alkylamido betaines, wherein the alkylamido betaines are derived from Macaúba palm oil.

In the following, preferred embodiments of the above composition are described in further detail. It is to be understood that each preferred embodiment is relevant on its own as well as in combination with other preferred embodiments.

In a first preferred embodiment A1 of the first aspect of the invention, the Macaúba palm oil is obtained by extraction of the fruits, palm pulp, and/or palm kernel of the Macaúba palm, preferably Acrocomia aculeata, preferably is extracted from the palm pulp and/or the palm kernel of the Macaúba palm, preferably Acrocomia aculeata, more preferably is extracted from the palm kernel of the Macaúba palm, preferably Acrocomia aculeata.

In a second embodiment A2 of the first aspect of the present invention, the composition comprises at least 45 wt.-%, based on the total weight of the composition, of C₄-C₂₂ alkylamido betaines, preferably C₆-C₂₀ alkylamido betaines, more preferably C₈-C₁₈ alkylamido betaines, even more preferably C₈-C₁₆ alkylamido betaines or C₁₆-C₁₈ alkylamido betaines, and in particular C₁₀-C₁₆ alkylamido betaines or C₁₂-C₁₄ alkylamido betaines.

In a third embodiment A3 of the first aspect of the present invention the alkylamido betaines are a mixture of compounds of the general formula (I)

R¹—CONH—X—N⁺R²R³—R⁴—Y (I)

wherein R¹ is an alkyl or alkenyl moiety containing 3 to 21 carbon atoms, preferably 7 to 19 carbon atoms, X is a (CH₂)ₙ group, wherein n is an integer from 1 to 6, R² and R³ independently are an alkyl moiety with 1 to 4 carbon atoms or an hydrogen atom, R⁴ is a C₁-alkylene, and Y is a COO⁻ group. Even more preferably, R² is identical to R³, most preferably R² and R³ each are a methyl group. Likewise, most preferably, X in formula (I) is a (CH₂)₃-group. Hence, preferably the alkylamido betaines are alkylamidopropyl betaines

In a fourth embodiment A4 of the first aspect of the present invention the composition comprises:
1 to 20 wt.-% of a C₈ alkylamido betaine, preferably alkylamido propyl betaine,
1 to 8 wt.-% of a C₁₀ alkylamido betaine, preferably alkylamido propyl betaine,
30 to 48 wt.-% of a C₁₂ alkylamido betaine, preferably alkylamido propyl betaine,
5 to 15 wt.-% of a C₁₄ alkylamido betaine, preferably alkylamido propyl betaine,
4 to 13 wt.-% of a C₁₆ alkylamido betaine, preferably alkylamido propyl betaine,
15 to 42 wt.-% of a C₁₈ alkylamido betaine, preferably alkylamido propyl betaine, and
0 to 5 wt.-% of a C₂₀ alkylamido betaine, preferably alkylamido propyl betaine,
each based on the total weight of the composition. Even more preferably, the composition comprises:
3 to 7 wt.-%, preferably 4 to 6 wt.-%, of a C₈ alkylamido betaine, preferably alkylamido propyl betaine,
2 to 6 wt.-%, preferably 3 to 5 wt.-%, of a C₁₀ alkylamido betaine, preferably alkylamido propyl betaine,
36 to 46 wt.-%, preferably 38 to 42 wt.-%, of a C₁₂ alkylamido betaine, preferably alkylamido propyl betaine,
6 to 13 wt.-%, preferably 8 to 11 wt.-%, of a C₁₄ alkylamido betaine, preferably alkylamido propyl betaine,
5 to 11 wt.-%, preferably 6 to 9 wt.-%, of a C₁₆ alkylamido betaine, preferably alkylamido propyl betaine,
25 to 40 wt.-%, preferably 30 to 38 wt.-% of a C₁₈ alkylamido betaine, preferably alkylamido propyl betaine, and
0 to 4 wt.-%, preferably 0 to 3 wt.-%, of a C₂₀ alkylamido betaine, preferably alkylamido propyl betaine,
each based on the total weight of the composition.

Preferably, in the composition of the present invention, the weight ratio of C₁₂ alkylamido betaine to a C₁₄ alkylamido betaine is from 77:23 to 87:13, preferably from 79:21 to 84:16

In a second aspect the present invention relates to a composition comprising alkylamido betaines, obtainable by a process comprising the steps of:
a) subjecting Macaúba palm oil or a fatty acid composition derived from Macaúba palm oil to a reaction with 3-dimethylaminopropyl amine yielding an intermediate; and
b) subjecting the intermediate to a reaction with chloroacetic acid or sodium mono-chloroacetate yielding the alkylamido betaines.

The compositions according to the first and the second aspect of the present invention have in particular better storage stability than compositions comprising alkylamido betaines derived from other oil sources. This results in less color formation during storage, especially during storage at elevated temperatures such as 40° C. or more.

In a third aspect the present invention relates to the use of a composition according to any the preceding embodiments of the first and the second aspect of the present invention as an amphoteric surfactant in a personal care composition, or cleaning composition.

In a fourth aspect the present invention relates to the use of the composition according to any of first and second aspects for personal care and cosmetic products in the form of: a body milk, creams, lotions, aftershave lotions, sprayable emulsions, tonics and scented waters, products for eliminating body odor such as deodorants and antiperspirants, make-up removers, conditioners, styling products, hair shampoos, shower gels, soaps, syndets, washing pastes, washing lotions, scrub preparations, foam baths, oil baths, shower baths, shaving foams, shaving lotions, shaving creams and dental care products.

In a fourth aspect the present invention relates to the use of the composition according to any of the first and second aspects for products with a low pH for cleaning hard surfaces, such as bath and toilet cleaners and the like, and also for cleaning and/or fragrance gels for use in sanitary installations.

In a fourth aspect the present invention relates to a personal care composition or a cleaning composition comprising a composition according to any of the preceding embodiments of the first and the second aspect of the present invention.

The compositions of the fourth aspect of the present invention have in particular better storage stability, application and performance properties than compositions comprising alkylamido betaines derived from other oil sources. This results in less color formation during storage, especially during storage at elevated temperatures such as 40° C. or more. Furthermore, these compositions have a better temperature dependency of viscosity. I.e., the temperature affects the viscosity less than for compositions derived from other oil sources. Furthermore, the viscosity and the flow behavior are also improved Finally, these compositions show a clearly improved foaming power in comparison to compositions derived from other oil sources

### BRIEF DESCRIPTION OF THE DRAWINGS

FIG. 1 shows the color of alkylamido propyl betaine compositions derived from palm kernel oil (PKO) and Macaúba palm oil (MAC) after three months of storage at 5° C., room temperature and 50° C.

FIG. 2 shows the color of shampoos and soaps comprising alkylamido propyl betaine compositions derived from palm kernel oil (PKO) and Macaúba palm oil (MAC) after three months of storage at 5° C., 25° C., and 45° C.

### DETAILED DESCRIPTION OF THE INVENTION

Before describing in detail exemplary embodiments of the present invention, definitions which are important for understanding the present invention are given.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±10%, preferably ±8%, more preferably ±5%, even more preferably ±2%. It is to be understood that the term “comprising” and “encompassing” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As used herein the term “does not comprise”, “does not contain”, or “free of” means in the context that the composition of the present invention is free of a specific compound or group of compounds, which may be combined under a collective term, that the composition does not comprise said compound or group of compounds in an amount of more than 0.8% by weight, based on the total weight of the composition. Furthermore, it is preferred that the composition according to the present invention does not comprise said compounds or group of compounds in an amount of more than 0.5% by weight, preferably the composition does not comprise said compounds or group of compounds at all.

When referring to compositions and the weight percent of the therein comprised ingredients it is to be understood that according to the present invention the overall amount of ingredients does not exceed 100% (±1% due to rounding).

The term “personal care composition” refers to any topical and oral product that can be used at least once daily by the consumer as an everyday care product for caring, cleaning, protecting, maintaining, perfuming or changing the appearance or feel of the human body, e.g., face, hair, skin or oral care. The personal care composition may comprise one or more active agents, e.g., organic and/or inorganic UV filters, as well as other ingredients or additives, e.g., emulsifiers, emollients, viscosity regulators, stabilizers, preservatives, or fragrances. Suitable daily care compositions are according to the present invention, e.g. leave-on face and body care products and rinse-off face, hair and body care products.

Suitable rinse-off products are hair shampoos, shower gels, soaps, syndet bars, washing pastes, washing lotions, scrub preparations, facial cleansers, intimate hygiene washes, foam baths, oil baths, shower baths, shaving foams, shaving lotions, shaving creams, foaming powders/tabs and dental care products (for example toothpastes, mouthwashes and the like). Also baby care products like baby shampoo and baths are suitable rinse-off products.

The term “sunscreen composition” or “sunscreen” refers to any topical product, which absorbs, and which may further reflect and scatter certain parts of UV radiation. Thus, the term “sunscreen composition” is to be understood as not only including sunscreen compositions, but also any cosmetic compositions that provide UV protection. The term “topical product” refers to a product that is applied to the skin and can refer, e.g., to sprays, lotions, creams, oils, foams, powders, or gels. According to the present invention the sunscreen composition may comprise one or more active agents, e.g., organic and inorganic UV filters, as well as other ingredients or additives, e.g., emulsifiers, emollients, viscosity regulators, stabilizers, preservatives, or fragrances.

Suitable decorative preparations are, e.g., lipsticks, nail varnishes, eye shadows, mascaras, dry and moist make-up, rouge, powders, depilatory agents and suntan lotions.

Suitable skin care preparations are e.g., moisturizing, refining, and lifting preparations. The cited daily care compositions can be in the form of creams, ointments, pastes, foams, gels, lotions, powders, make-ups, sprays, sticks or aerosols.

The term “UV filter” or “ultraviolet filter” as used herein refers to organic or inorganic compounds, which can absorb and may further reflect and scatter UV radiation caused by sunlight. UV-filter can be classified based on their UV protection curve as UV-A, UV-B, or broadband filters.

In general, UV light can be divided into UV-A radiation (320-400 nm) and UV-B radiation (290-320 nm). The definition of “broadband” protection (also referred to as broad-spectrum or broad protection) is based on the “critical wavelength”. For broadband coverage, UV-B and UV-A protection must be provided. According to the US requirements, a critical wavelength of at least 370 nm is required for achieving broad spectrum protection. The term “critical wavelength” is defined as the wavelength at which the area under the UV protection curve (% protection versus wavelength) represents 90% of the total area under the curve in the UV region (290-400 nm). For example, a critical wavelength of 370 nm indicates that the protection of the sunscreen composition is not limited to the wavelengths of UV-B, i.e., wavelengths from 290-320 nm, but extends to 370 nm in such a way that 90% of the total area under the protective curve in the UV region are reached at 370 nm.

The term “emollient” relates to cosmetic specific oils used for protecting, moisturizing and lubricating the skin. The word emollient is derived from the Latin word mollire, to soften. In general, emollients prevent evaporation of water from the skin by forming an occlusive coating. They can be divided into different groups depending on their polarity index.

The term “polarity index” refers to non-polar or polar oils. Non-polar oils are mainly based on hydrocarbons and lack an electronegative element, such as oxygen. In contrast, polar oils contain heteroatoms that differ in electronegativity, which results in a dipole moment. However, such oils are still insoluble in water, i.e., hydrophobic. The polarity index can be determined by measuring the interfacial tension between the respective oil and water.

The term “fatty acid” as used herein is directed to linear or branched, preferably linear, primary carboxylic acids. Fatty acids may comprise from 4 to 26 carbon atoms. According to the present invention, the term fatty acid encompasses saturated and unsaturated acids. The double bond of an unsaturated fatty acid can give either cis or trans isomers. Caprylic acid, capric acid, lauric acid, myristic acid, myristoleic acid, palmitic acid, palmitoleic acid, sapienic acid, stearic acid, oleic acid, elaidic acid, vaccenic acid, linoleic acid, linoelaidic acid, α-Linolenic acid, arachidic acid, arachidonic acid, eicosapentaenoic acid, erucic acid, behenic acid, docosahexaenoic acid, lignoceric acid, and cerotic acid should be named in this connection.

The prefix Cₙ-Cₘ indicates in each case the possible number of carbon atoms in the group.

The term “alkyl” as used herein denotes in each case a linear or branched alkyl group having usually from 1 to 30 carbon atoms, preferably 4 to 26 or of 1 to 6 or of 1 to 3 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “alkoxy” as used herein denotes in each case a linear or branched alkyl group which is bonded via an oxygen atom and has usually from 1 to 6 carbon atoms, preferably 1 to 2 carbon atoms, more preferably 1 carbon atom. Examples of an alkoxy group are methoxy, ethoxy, n-propoxy, iso-propoxy, n-butyloxy, 2-butyloxy, iso-butyloxy, tert.-butyloxy, and the like.

As used herein, the term “alkylene” refers to a linking linear or branched alkylene group having usually from 1 to 4 carbon atoms, e.g., 1, 2, 3, or 4 carbon atoms. The alkylene group bridges a certain group to the remainder of the molecule. Preferred alkylene groups include methylene (CH₂), ethylene (CH₂CH₂), propylene (CH₂CH₂CH₂) and the like. A skilled person understands that, if it is referred, e.g., to CH₂ that the carbon atom being tetravalent has two valences left for forming a bridge (—CH₂—). Similarly, when it is referred, e.g., to CH₂CH₂, each carbon atom has one valence left for forming a bridge (—CH₂CH₂—). Furthermore, when it is referred, e.g., to CH₂CH₂CH₂, each terminal carbon atom has one valence left for forming a bridge (—CH₂CH₂CH₂—).

Further, a skilled person is aware that resonance structures of the oxidized forms may be possible. Saturated heterocycles include, unless otherwise indicated, in general 3- to 9-membered, preferably 4- to 8-membered or 5- to 7-membered, more preferably 5- or 6-membered monocyclic rings comprising 3 to 9, preferably 4 to 8 or 5 to 7, more preferably 5 or 6 atoms comprising at least one heteroatom, such as pyrrolidine, tetrahydrothiophene, tetrahydrofuran, piperidine, tetrahydropyran, dioxane, morpholine or piperazine.

The term “aryl” or “aromatic carbocycle” preferably includes 6-membered aromatic carbocyclic rings based on carbon atoms as ring members. A preferred example is phenyl.

The term “oil palm” as used herein denotes a species of palm, which is also known as “Elaeis guineensis”. It is the principal source of “palm oil”.

The term “coconut tree” as used herein denotes a member of the palm tree family (Arecaceae) and is also referred to as Cocos nucifera. It is the principal source for “coconut oil”.

The term “Macaúba palm” as used herein denotes a species of palm. Exemplary species are known as “Acrocomia aculeata” (also known as “macaiba”, “boicaiuva”, “macaúva”, “coco-de-catarro”, “coco-baboso”, and “coco-de-espinho”), “Acrocomia hassleri”, and “Acrocomia totei”. Macaúba palms can grow high, e.g., up to about 15 m. The Macaúba fruit comprises pulp and kernel.

The term “pulp” as used herein refers to inner flesh of a fruit.

The term “kernel” as used herein is interchangeable with “seed” or “almond”.

The term “cleaning composition” as used herein encompasses home care formulation, industrial care formulation, and institutional care formulation. Home care formulations are typically used by private consumers, whereas industrial care formulations are typically used by the industry, and institutional care formulations are typically used in e.g., clinics and nursing homes. It is however also possible that the respective formulations can be used in different areas than intended. Hence, the institutional care formulation may also be used by private consumers or the industry and vice versa. Typically cleaning compositions are e.g., for the laundry, dishwashing, hard surface cleaning, food service and kitchen hygiene, food and beverage processing, commercial laundry, sanitation, institutional cleaning, industrial cleaning, and vehicle and transportation care.

The term “liquid” as used herein also encompasses semi-solid conditions, wherein the fluid has an increased viscosity (e.g., creamy, gels, ointments).

The term “crop formulation” as used herein encompasses pesticide formulations, fungicide formulations, and herbicide formulations.

The term “oil yield in tons per hectare per year” as used herein is directed to the oil derived from the fruit of the plant via e.g., extraction, wherein the fruit comprises the pulp and the kernel. It refers to the oil produced per hectare. It is to be understood that the value refers to the oil yield obtained from a monoculture, wherein the plants are cultivated under standard conditions, which depend on the respective plant and are known to the skilled person. Hence, in the event that the plant is not cultivated in a monoculture (e.g., on a cattle field), the respective value for this particular cultivation may be reduced. Typically, oil palm has an oil yield in tons per hectare per year of about 3.8 t/ha/yr, rapeseed has an oil yield in tons per hectare per year of about 0.8 t/ha/yr, sunflower has an oil yield in tons per hectare per year of about 0.7 t/ha/yr, and soya has an oil yield in tons per hectare per year of about 0.6 t/ha/yr.

The term “monoculture” as used herein denotes the practice of growing one plant, e.g., Macaúba palm, in a field at a time. On the example of Macaúba palm, about 500 to about 600 palms can be planted per hectare. In this connection, it is preferred that the minimum distance between the tress is about 3.5 to 4.5 meters. This number varies depending on e.g., the soil. The growing of the Macaúba plants is described in the following. In the first year, growth is slower, as the major development occurs below the soil. Hence, the plant itself grows about 80 to 100 cm. From the second year onwards, when the plant size is approximately 100 to 150 cm), growth is faster and there is an increased development of the aerial part of the plant. A fully mature plant providing the claimed oil yield per hectare per year is about 5 to 6 years old.

The water consumption of the Macaúba plant is 50% lower than of palm. Macaúba plantations can be located in regions with a minimum rainfall of 1.200 mm per year.

The term “amphoteric” as used herein means that the compound contains an acidic and a basic moiety.

The term “amphoteric surfactants” as used herein denotes a class of surfactants. Best known and widely used are the so-called alkylamido betaines, especially alkylamido propyl betaine. These betaines are extensively used in personal care products, as shampoos, bubble baths and other cleansing products, because they are considered as being very mild while having high foaming capacities.

The term “agroforestry” as used herein denotes a land use management system in which trees or shrubs are grown around or among other plant such as other trees or other shrubs or crops or pastureland. It is to be understood that not only one further plant can be present in agroforestry. On the example of Macaúba palm, e.g., about 250 to about 360 or about 325 to about 350, trees can be planted per hectare. In this connection, suitable crops that may be planted together with Macaúba palm are exemplarily beans, mandioca, corn, cereals, sunflower, peanut, rapeseed, soya, and mixtures thereof.

The term “silvopastoral” as used herein denotes a land use management system in which trees and optionally forage are planted within the grazing of domesticated animals. On the example of Macaúba palm, e.g., about 275 to about 450 or about 375 to about 400, trees can be planted per hectare.

One advantage of the present invention is that the compositions comprising alkylamido betaines of the present invention exhibit improved storage stability and improved handling and processability.

The compositions comprising alkylamido betaines of the present invention have in particular an improved storage stability. Improved storage stability is achieved if the composition does not exhibit any visible (e.g., cloudiness, discoloration, phase separation, agglomeration) or measurable (e.g. pH, viscosity, active substance content, color value) or perceivable (odor) changes over time. Preferably, improved storage stability is achieved if less color formation during storage at elevated temperatures such as 40° C. or more is observed. Also preferably, improved storage stability is achieved if no cloudiness or phase separation during storage at low temperatures such as 15° C. or lower can be observed in case of aqueous compositions. Even more preferably, improved storage stability is achieved if the composition solidifies and melts over a well-defined, narrow temperature range at a low temperature level (<15° C.). The solidification-/melting-temperature range of a composition can be determined by a DSC measurement (differential scanning calorimetry). Most preferably, improved storage stability is achieved if the time wherein the composition is unchanged and stable is as long as possible. In particular improved storage stability is achieved for dried compositions comprising alkylamido betaines of the present invention such as powders, if no agglomerating, blocking or lumping during storage at elevated temperatures such as 40° C. or more is observed and/or if the composition has a good microbiological stability (no growth of microorganisms), e.g. acc. to requirements of Ph.Eur. 5.1.3 or DIN EN ISO 11930.

Preferably, the compositions comprising alkylamido betaines of the present invention have an improved handling and processability. Improved handling and processability is in particular achieved if the aqueous composition has a viscosity that allows for free-flowing at room temperature (˜23° C.) and/or to be easily stirred and pumped (e.g. showing a shear thinning behavior). Preferably, improved handling and processability is achieved if the aqueous composition can be dissolved rapidly in a water phase, ideally without additional heating (at ˜23° C.). Also preferably, improved handling and processability is achieved if the time that is needed to prepare a personal care composition with this composition is as short as possible. Thus, most preferably, improved handling and processability is achieved if dried compositions comprising alkylamido betaines of the present invention (e.g., powder) easily disintegrate and dissolve in water and/or if dried compositions (e.g. powder) are suitable to be incorporated into solid personal care compositions, i.e. suitable for a extrusion or pelletising process.

One advantage of the present invention is that the personal care compositions comprising alkylamido betaines of the present invention show improved viscosity and flow behavior and/or improved appearance/transparency and/or improved the mildness and/or improved surface activity and cleaning capability and/or improved foaming capability and/or improved care performance.

What is understood as advantageous viscosity and flow behavior depends on the intended use and application of the personal care compositions. For aqueous personal care compositions comprising alkylamido betaines of the present invention such as shower gels and shampoos it is desirable to have a high viscosity in the range of 5000-20000 mPas (measured using a Brookfield RV laboratory rheometer at 23° C., 10 rpm, spindle choice depending on viscosity range). As is known, “mPas” means millipascal seconds. Hence, improved viscosity and flow behavior for aqueous personal care compositions comprising alkylamido betaines of the present invention such as shower gels and shampoos is preferably achieved by either having higher viscosity with the same concentration of alkylamido betaine (and same concentration of other components) or by having the same viscosity with a lower concentration of alkylamido betaine (and/or lower concentration of other components). Preferably, improved viscosity and flow behavior for aqueous personal care compositions comprising alkylamido betaines of the present invention such as shower gels and shampoos is achieved, if the viscosity is not or less sensitive to the temperature (e.g., no or less viscosity decrease at elevated temperatures such as 40° C. or more). Finally, also preferably, improved viscosity and flow behavior for aqueous personal care compositions comprising alkylamido betaines of the present invention such as shower gels and shampoos is achieved if the viscosity is not or less sensitive to the addition of a parfum (e.g. no or less viscosity decrease), and/or if the compositions have a shear-thinning flow behavior, means that the viscosity decreases when shear-stress is applied to the composition (e.g. when it's moved, stirred, pumped, shaken), and/or if the viscosity when the composition is at rest (in the bottle/packaging, when collected on the palm of hand) is high and the viscosity under shear-stress (e.g. when dosed, squeezed out of the bottle or distributed/applied and rubbed between hands and/or hair) is low.

For aqueous personal care compositions comprising alkylamido betaines of the present invention such as pump foams, sprays, micellar waters, or make-up removers a very low viscosity of less than 200 mPas is desired. In this case it is advantageous to either achieve a lower viscosity with the same concentration of alkylamido betaine (and same concentration of other components) or the same viscosity with a higher concentration of alkylamido betaine (and/or higher concentration of other components).

For aqueous personal care compositions comprising alkylamido betaines of the present invention very often clear compositions are required, especially if transparent packaging is used. Hence, preferably, the care compositions of the present invention have an improved transparency. Transparency can be quantitatively determined by means of a transmission measurement with a TurbiScan MA 2000 (measuring instrument from Formulaction) at 23° C. Preferably, the care composition of the present invention has an average transmission of at least 80%, preferably more of at least 85%, and most preferably of at least 88%.

For personal care compositions comprising alkylamido betaines of the present invention it is desirable to have a good skin and mucous membrane (ocular and oral) compatibility and thus a low irritation potential, also denoted as mildness. The personal care compositions comprising alkylamido betaines of the present invention have an improved mildness. The irritation potential can be determined by methods known to those skilled in the art, e.g., in vitro methods like RBC, HET-CAM or test on model tissues (Epi-Ocular/Epi-Oral) and also by test subjects (e.g., epicutaneous patch test, “tear-free” tests with panelist).

For personal care compositions comprising alkylamido betaines of the present invention it is also advantageous to have improved surface activity. Improved surface activity is characterized by either a lower CMC (critical micellar concentration), a lower surface tension or the ability to reduce the surface tension faster than a composition comprising alkylamido betaines derived from other oil sources. The surface can for example be the interface between the aqueous composition and either air, gas or oil. Hence, the personal care compositions comprising alkylamido betaines of the present invention preferably have an improved surface activity. The surface activity can be measured CMC measurement with a tensiometer (DCAT, DataPhysics Instruments GmbH or comparable), dynamic surface tension measurement with the bubble pressure method (SITA-Online T60, Sita Messtechnik GmbH or comparable). These characteristics are also relevant to determine the cleansing, dispersing, emulsifying and solubilisation capability of a composition, specifically for the removal of dirt and oily substances from hair, skin, hard surfaces, and textiles.

In parallel to the compositions comprising alkylamido betaines of the present invention, also the personal care compositions comprising alkylamido betaines of the present invention show improved storage stability as defined above.

It is desirable that personal care compositions comprising alkylamido betaines of the present invention have a good foaming ability. It is in particular desirable that the compositions are capable to create a foam with a high initial foam volume when the composition is used, e.g. by rubbing between hands, skin and/or hair; and/or that is stable after foam formation was finished, so showing no or less volume decrease compared to the intial foam volume and/or with no or less drainage (no or only little liquid accumulates below the foam); and/or that has small foam bubbles and a narrow foam bubble size distribution; and/or that has high water content (liquid volume in foam/total volume of foam); and/or that has high elasticity; and/or that has a pleasant foam sensory such as soft and dense feeling of the foam, creaminess, elasticity, moldability and overall acceptance by the consumer. Preferably, the foam derived from the personal care compositions comprising alkylamido betaines of the present invention shows the above-mentioned characteristics even under challenging conditions such as in hard water (e.g. 15° dH), at low temperatures, under acidic or alkaline conditions, in presence of a high oil load, in presence of a high salt load, with no anionic sulfate-surfactants present.

The foaming behavior of an aqueous composition (or a dried composition that has been dissolved in water) can be investigated e.g. by agitating the solution within a short time period (˜10-200 seconds) by means of stirring, shaking, pumping, bubbling through a gas stream or in other way and then monitoring the foam volume over time (up to 30 minutes) and taking pictures of the foam structure for image analysis. Test equipment such as the Foam Expert (SITA Messtechnik GmbH) or Dynamic Foam Analyzer DFA 100 (Krüss) can be used for that purpose. In addition, the foam can be evaluated by the means of rheological measurements in a viscosimeter.

Preferably, the personal compositions comprising alkylamido betaines derived from Macaúba oil leave a more caring feeling after use on hair and/or skin than compositions comprising alkylamido betaines derived from other oil sources. This can be investigated, for example, by a test panel by reference to subjective skin feel (smoothness, dryness etc.) or haptics and feel of the treated hair. Mechanical measurement methods, such as combability of the hair or hair breakage, can also be used.

Preferred embodiments regarding the composition comprising alkylamido betaines derived from Macaúba palm oil or the composition comprising alkylamido betaines obtainable by a process comprising the steps of amidation and reaction with chloroacetic acid, as well as the use thereof for the production of alkylamido betaines, the use thereof as surfactants in personal care compositions and/or cleaning compositions and these personal care composition and cleaning composition themselves are described hereinafter. It is to be understood that the preferred embodiments of the invention are preferred alone or in combination with each other.

It is a special advantage of the process of the present invention that it is more efficient and less energy intensive and thus has higher sustainability. Reason is that the Macaúba oil has a lower melting point than for example usually used palm kernel oil. Hence, it has lower viscosities at comparable temperatures and thus can be process with lower energy demand at lower temperatures. Another reason is that the sustainability of the Macaúba oil is higher than the substainability of oil from other oil sources such as coconut oil, palm oil, or palm kernel oil due to the less water intensive growing conditions, less need for space, higher productivity and higher robustness to allow for growing in less specialized conditions.

As indicated above, the present invention relates in one embodiment to a composition comprising alkylamido betaines derived from Macaúba palm oil.

Preferably, Macaúba palm oil is obtained by extraction of the fruits, palm pulp, and/or palm kernel of the Macaúba palm. In a preferred embodiment, the Macaúba palm is Acrocomia hassleri, Acrocomia totei, and/or Acrocomia aculeata, and in particular Acrocomia aculeata.

In a preferred embodiment, the Macaúba palm oil is extracted from the Macaúba kernel, preferably wherein the Macaúba palm is Acrocomia hassleri, Acrocomia totei, and/or Acrocomia aculeata and the oil is extracted from more preferably Acrocomia hassleri kernel, Acrocomia totei kernel, and/or Acrocomia aculeata kernel, and in particular wherein the Macaúba palm is Acrocomia aculeata and the oil is extracted from Acrocomia aculeata kernel.

In another preferred embodiment, the Macaúba palm oil is extracted from the Macaúba pulp, and in particular wherein the Macaúba palm is Acrocomia aculeata and the oil is extracted from Acrocomia aculeata pulp.

In another preferred embodiment, the Macaúba palm oil is extracted from the Macaúba pulp and kernel, and in particular wherein the Macaúba palm is Acrocomia aculeata and the oil is extracted from Acrocomia aculeata pulp and kernel.

In a preferred embodiment, the Macaúba palm can sufficiently grow under tropical and subtropical conditions.

In a preferred embodiment, the Macaúba palm can sufficiently grow in regions from the 30th parallel north to the 28th parallel south, preferably from the 25th parallel north to the 25th parallel south.

In a preferred embodiment, the Macaúba palm sufficiently grows at a temperature range of 18 to 30° C., more preferably of 20 to 28° C. In this connection it is to be understood that the temperature range is the average temperature over one year. Hence, the Macaúba palm is preferably less vulnerable to temperature fluctuation.

The term “sufficiently grow” as used herein denotes that the claimed oil yield is achievable under standard cultivation.

In addition, particularly oil palms need tropical conditions and preferred temperatures between about 24 to 28° C., monthly rainfalls of at least 100 mm/m², and a humidity between about 50 to 70%. These factors limit the possibility of a profitable cultivation.

In a preferred embodiment, the Macaúba palm provides a reduced water demand.

In a preferred embodiment, cultivating the Macaúba palm provides a reduction of the loss of biodiversity.

In a preferred embodiment, cultivating the Macaúba palm provides a reduction of loss of habitats for local tribes.

In a preferred embodiment, cultivating the Macaúba palm provides a reduction of deforestation.

In a preferred embodiment, cultivating the Macaúba palm provides an improved recovery of degraded areas and/or springs and watersheds.

In a preferred embodiment, the cultivating Macaúba palm provides an improved retention of moisture in the soil.

In this connection it is to be understood that the above-outlined reductions or improvements are compared to plants, in particular palms, having an oil yield in tons per hectare per year of less than 6 t/ha/yr, preferably compared to the Macaúba palm.

In a preferred embodiment, the Macaúba palm oil is the crude oil, i.e. not further treated after the extraction from the Macaúba palm.

In another preferred embodiment, the Macaúba palm oil is the filtered oil, i.e, wherein the crude oil is first filtered by any known in the art filtering systems and then used in the process. A suitable filtration process is e.g., press filtration.

In a preferred embodiment, the composition comprising alkylamido betaines comprises at least 45 wt.-% based on the total weight of the composition comprising alkylamido betaines, of C₄-C₂₂ alkylamido betaines, preferably alkylamido propyl betaines, preferably C₆-C₂₀ alkylamido betaines, preferably alkylamido propyl betaines, more preferably C₈-C₁₈ alkylamido betaines, preferably alkylamido propyl betaines, even more preferably C₈-C₁₆ alkylamido betaines, preferably alkylamido propyl betaines or C₁₆-C₁₈ alkylamido betaines, preferably alkylamido propyl betaines, and in particular C₁₀-C₁₆ alkylamido betaines, preferably alkylamido propyl betaines or C₁₂-C₁₄ alkylamido betaines, preferably alkylamido propyl betaines.

In a preferred embodiment, the composition comprising alkylamido betaines comprises at least 85 wt.-% based on the total weight of the composition comprising alkylamido betaines, of C₄-C₂₂ alkylamido betaines, preferably alkylamido propyl betaines, preferably C₁₀-C₂₂ alkylamido betaines, preferably alkylamido propyl betaines, more preferably C₁₂-C₂₀ alkylamido betaines, preferably alkylamido propyl betaines, even more preferably C₁₂-C₂₀ alkylamido betaines, preferably alkylamido propyl betaines, and in particular C₁₂-C₁₈ alkylamido betaines, preferably alkylamido propyl betaines.

In a preferred embodiment, the composition comprising alkylamido betaines comprises at least 10 wt.-% of C₁₆ alkylamido betaines, preferably alkylamido propyl betaines and at least 75 wt.-% of C₁₈ alkylamido betaines, preferably alkylamido propyl betaines, each based on the total weight of the composition comprising alkylamido betaines.

In a preferred embodiment, the composition comprising alkylamido betaines comprises 10 to 25 wt.-% of C₁₆ alkylamido betaines, preferably alkylamido propyl betaines and 75 to 90 wt.-% of C₁₈ alkylamido betaines, preferably alkylamido propyl betaines, each based on the total weight of the composition comprising alkylamido betaines.

In a preferred embodiment, the composition comprising alkylamido betaines comprises at least 80 wt.-%, preferably at least 90 wt.-%, and in particular at least 95 wt.-%, based on the total weight of the composition comprising alkylamido betaines, of C₁₂-C₁₄ alkylamido betaines, preferably lauramido propyl betaines.

In a preferred embodiment, the composition comprising alkylamido betaines comprises at least 80 wt.-%, preferably at least 90 wt.-%, and in particular at least 95 wt.-%, based on the total weight of the composition comprising alkylamido betaines, of C₁₂-C₁₈ alkylamido betaines, preferably alkylamido propyl betaines.

In a preferred embodiment, the composition comprising alkylamido betaines comprises at least 2 wt.-% of C₁₀ alkylamido betaines, preferably alkylamido propyl betaines, at least 35 wt.-% of C₁₂ alkylamido betaines, preferably alkylamido propyl betaines, at least 5 wt.-% of C₁₄ alkylamido betaines, preferably alkylamido propyl betaines, and at least 4 wt.-% of C₁₆ alkylamido betaines, preferably alkylamido propyl betaines, each based on the total weight of the composition comprising alkylamido betaines.

In a preferred embodiment, the composition comprising alkylamido betaines comprises 3 to 7 wt.-% of C₈ alkylamido betaines, preferably alkylamido propyl betaines, 2 to 6 wt.-% of C₁₀ alkylamido betaines, preferably alkylamido propyl betaines, 35 to 45 wt.-% of C₁₂ alkylamido betaines, preferably alkylamido propyl betaines, 5 to 13 wt.-% of C₁₄ alkylamido betaines, preferably alkylamido propyl betaines, and 4 to 10 wt.-% of C₁₆ alkylamido betaines, each based on the total weight of the composition comprising alkylamido betaines.

In a preferred embodiment, the composition comprising alkylamido betaines comprises at least 90 wt.-% of C₈ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₀ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₂ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₄ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₆ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₈ alkylamido betaines, preferably alkylamido propyl betaines, based on the total weight of the composition comprising alkylamido betaines.

In a preferred embodiment, the Macaúba palm has an oil yield in tons per hectare per year in the range of at least 7 t/ha/yr, preferably at least 8 t/ha/yr.

In a preferred embodiment, the Macaúba palm has an oil yield in tons per hectare per year in the range of 6 to 30 t/ha/yr, preferably 7 to 20 t/ha/yr, more preferably of 8 to 15 t/ha/yr or of 8 to 12 t/ha/yr or of 8 to 11 t/ha/yr.

In a preferred embodiment, the composition comprising alkylamido betaines comprises
1 to 20 wt.-% of a C₈ alkylamido betain, preferably alkylamidoalkylamido propyl betain,
1 to 8 wt.-% of a C₁₀ alkylamido betain, preferably alkylamidoalkylamido propyl betain,
30 to 48 wt.-% of a C₁₂ alkylamido betain, preferably alkylamidoalkylamido propyl betain,
5 to 15 wt.-% of a C₁₄ alkylamido betain, preferably alkylamidoalkylamido propyl betain,
4 to 13 wt.-% of a C₁₆ alkylamido betain, preferably alkylamidoalkylamido propyl betain,
15 to 42 wt.-% of a C₁₈ alkylamido betain, preferably alkylamidoalkylamido propyl betain, and
0 to 5 wt.-% of a C₂₀ alkylamido betain, preferably alkylamidoalkylamido propyl betain,
each based on the total weight of the composition comprising alkylamido betaines. Said composition comprising alkylamido betaines is preferably obtained from oil extracted from Macaúba kernel.

In a preferred embodiment, the composition comprising alkylamido betaines comprises
3 to 7 wt.-%, preferably 4 to 6 wt.-%, of a C₈ alkylamido betain, preferably alkylamidoalkylamido propyl betain,
2 to 6 wt.-%, preferably 3 to 5 wt.-%, of a C₁₀ alkylamido betain, preferably alkylamidoalkylamido propyl betain,
36 to 46 wt.-%, preferably 38 to 42 wt.-%, of a C₁₂ alkylamido betain, preferably alkylamidoalkylamido propyl betain,
6 to 13 wt.-%, preferably 8 to 11 wt.-%, of a C₁₄ alkylamido betain, preferably alkylamidoalkylamido propyl betain,
5 to 11 wt.-%, preferably 6 to 9 wt.-%, of a C₁₆ alkylamido betain, preferably alkylamidoalkylamido propyl betain,
25 to 40 wt.-%, preferably 30 to 38 wt.-% of a C₁₈ alkylamido betain, preferably alkylamidoalkylamido propyl betain, and
0 to 4 wt.-%, preferably 0 to 3 wt.-%, of a C₂₀ alkylamido betain, preferably alkylamidoalkylamido propyl betain,
each based on the total weight of the composition comprising alkylamido betaines. Said composition comprising alkylamido betaines is preferably obtained from oil extracted from Macaúba kernel.

In a preferred embodiment, the composition comprising alkylamido betaines comprises
0 to 5 wt.-%, preferably 0 to 3 wt.-%, and in particular 0 to 2 wt.-%, of a C₁₀ alkylamido betain, preferably alkylamidoalkylamido propyl betain,
0 to 6 wt.-%, preferably 0 to 5 wt.-%, and in particular 1 to 4 wt.-%, of a C₁₂ alkylamido betain, preferably alkylamidoalkylamido propyl betain,
0 to 6 wt.-%, preferably 0 to 5 wt.-%, and in particular 1 to 4 wt.-%, of a C₁₄ alkylamido betain, preferably alkylamidoalkylamido propyl betain,
10 to 35 wt.-%, preferably 13 to 32 wt.-%, and in particular 15 to 30 wt.-%, of a C₁₆ alkylamido betain, preferably alkylamidoalkylamido propyl betain,
55 to 85 wt.-%, preferably 60 to 80 wt.-%, and in particular 65 to 75 wt.-%, of a C₁₈ alkylamido betain, preferably alkylamidoalkylamido propyl betain,
0 to 4 wt.-%, preferably 0 to 3 wt.-%, and in particular 0 to 2 wt.-%, of a C₂₀ alkylamido betain, preferably alkylamidoalkylamido propyl betain,
each based on the total weight of the composition comprising alkylamido betaines. Said composition comprising alkylamido betaines is preferably obtained from oil extracted from Macaúba pulp.

In a preferred embodiment, the composition comprising alkylamido betaines comprises 0.1 to 10 wt.-% of a C₆ alkylamido betain, preferably cocamidoalkylamido betain, more preferably cocamidoalkylamido propyl betain,
1 to 20 wt.-% of a C₈ alkylamido betain, preferably cocamidoalkylamido betain, more preferably cocamidoalkylamido propyl betain,
1 to 8 wt.-% of a C₁₀ alkylamido betain, preferably cocamidoalkylamido betain, more preferably cocamidoalkylamido propyl betain,
30 to 48 wt.-% of a C₁₂ alkylamido betain, preferably cocamidoalkylamido betain, more preferably cocamidoalkylamido propyl betain,
5 to 15 wt.-% of a C₁₄ alkylamido betain, preferably cocamidoalkylamido betain, more preferably cocamidoalkylamido propyl betain,
4 to 13 wt.-% of a C₁₆ alkylamido betain, preferably cocamidoalkylamido betain, more preferably cocamidoalkylamido propyl betain,
15 to 42 wt.-% of a C₁₈ alkylamido betain, preferably cocamidoalkylamido betain, more preferably cocamidoalkylamido propyl betain, and
0 to 5 wt.-% of a C₂₀ alkylamido betain, preferably cocamidoalkylamido betain, more preferably cocamidoalkylamido propyl betain,
each based on the total weight of the composition comprising alkylamido betaines. Said composition comprising alkylamido betaines is preferably obtained from oil extracted from Macaúba kernel.

In a preferred embodiment, the composition comprising alkylamido betaines comprises 0.2 to 4 wt.-%, preferably 0.4 to 1.5 wt.-%, of a C₆ alkylamido betain, preferably cocamidoalkylamido betain, more preferably cocamidoalkylamido propyl betain,
3 to 7 wt.-%, preferably 4 to 6 wt.-%, of a C₈ alkylamido betain, preferably cocamidoalkylamido betain, more preferably cocamidoalkylamido propyl betain,
2 to 6 wt.-%, preferably 3 to 5 wt.-%, of a C₁₀ alkylamido betain, preferably cocamidoalkylamido betain, more preferably cocamidoalkylamido propyl betain,
36 to 46 wt.-%, preferably 38 to 42 wt.-%, of a C₁₂ alkylamido betain, preferably cocamidoalkylamido betain, more preferably cocamidoalkylamido propyl betain,
6 to 13 wt.-%, preferably 8 to 11 wt.-%, of a C₁₄ alkylamido betain, preferably cocamidoalkylamido betain, more preferably cocamidoalkylamido propyl betain,
5 to 11 wt.-%, preferably 6 to 9 wt.-%, of a C₁₆ alkylamido betain, preferably cocamidoalkylamido betain, more preferably cocamidoalkylamido propyl betain, to 40 wt.-%, preferably 30 to 38 wt.-% of a C₁₈ alkylamido betain, preferably cocamidoalkylamido betain, more preferably cocamidoalkylamido propyl betain, and
0 to 4 wt.-%, preferably 0 to 3 wt.-%, of a C₂₀ alkylamido betain, preferably cocamidoalkylamido betain, more preferably cocamidoalkylamido propyl betain,
each based on the total weight of the composition comprising alkylamido betaines. Said composition comprising alkylamido betaines is preferably obtained from oil extracted from Macaúba kernel.

In a preferred embodiment, the composition comprising alkylamido betaines comprises at least 45 wt.-% based on the total weight of the composition comprising alkylamido betaines, of C₄-C₂₂ alkylamido betaines, preferably alkylamido propyl betaines, preferably C₆-C₂₀ alkylamido betaines, preferably alkylamido propyl betaines, more preferably C₈-C₁₈ alkylamido betaines, preferably alkylamido propyl betaines, even more preferably C₈-C₁₆ alkylamido betaines, preferably alkylamido propyl betaines or C₁₆-C₁₈ alkylamido betaines, preferably alkylamido propyl betaines, and in particular C₁₀-C₁₆ alkylamido betaines, preferably alkylamido propyl betaines or C₁₂-C₁₄ alkylamido betaines, preferably alkylamido propyl betaines.

In a preferred embodiment, the composition comprising alkylamido betaines comprises at least 85 wt.-% based on the total weight of the composition comprising alkylamido betaines, of C₄-C₂₂ alkylamido betaines, preferably alkylamido propyl betaines, preferably C₁₀-C₂₂ alkylamido betaines, preferably alkylamido propyl betaines, more preferably C₁₂-C₂₀ alkylamido betaines, preferably alkylamido propyl betaines, even more preferably C₁₂-C₂₀ alkylamido betaines, preferably alkylamido propyl betaines, and in particular C₁₂-C₁₈ alkylamido betaines, preferably alkylamido propyl betaines.

In a preferred embodiment, the composition comprising alkylamido betaines comprises at least 10 wt.-% of C₁₆ alkylamido betaines, preferably alkylamido propyl betaines and at least 75 wt.-% of C₁₈ alkylamido betaines, preferably alkylamido propyl betaines, each based on the total weight of the composition comprising alkylamido betaines.

In a preferred embodiment, the composition comprising alkylamido betaines comprises 10 to 25 wt.-% of C₁₆ alkylamido betaines, preferably alkylamido propyl betaines and 75 to 90 wt.-% of C₁₈ alkylamido betaines, preferably alkylamido propyl betaines, each based on the total weight of the composition comprising alkylamido betaines.

In a preferred embodiment, the composition comprising alkylamido betaines comprises at least 80 wt.-%, preferably at least 90 wt.-%, and in particular at least 95 wt.-%, based on the total weight of the composition comprising alkylamido betaines, of C₁₂-14 alkylamido betaines, preferably alkylamido propyl betaines.

In a preferred embodiment, the composition comprising alkylamido betaines comprises at least 80 wt.-%, preferably at least 90 wt.-%, and in particular at least 95 wt.-%, based on the total weight of the composition comprising alkylamido betaines, of C₁₂-18 alkylamido betaines, preferably alkylamido propyl betaines.

In a preferred embodiment, the composition comprising alkylamido betaines comprises at least 2 wt.-% of C₁₀ alkylamido betaines, preferably alkylamido propyl betaines, at least 35 wt.-% of C₁₂ alkylamido betaines, preferably alkylamido propyl betaines, at least 5 wt.-% of C₁₄ alkylamido betaines, preferably alkylamido propyl betaines, and at least 4 wt.-% of C₁₆ alkylamido betaines, preferably alkylamido propyl betaines, each based on the total weight of the composition comprising alkylamido betaines.

In a preferred embodiment, the composition comprising alkylamido betaines comprises 3 to 7 wt.-% of C₈ alkylamido betaines, preferably alkylamido propyl betaines, 2 to 6 wt.-% of C₁₀ alkylamido betaines, preferably alkylamido propyl betaines, 35 to 45 wt.-% of C₁₂ alkylamido betaines, preferably alkylamido propyl betaines, 5 to 13 wt.-% of C₁₄ alkylamido betaines, preferably alkylamido propyl betaines, and 4 to 10 wt.-% of C₁₆ alkylamido betaines, preferably alkylamido propyl betaines, each based on the total weight of the composition comprising alkylamido betaines.

In a preferred embodiment, the composition comprising alkylamido betaines comprises at least 90 wt.-% of C₈ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₀ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₂ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₄ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₆ alkylamido betaines, preferably alkylamido propyl betaines, based on the total weight of the composition comprising alkylamido betaines.

In a preferred embodiment, the composition comprising alkylamido betaines comprises at least 90 wt.-% of C₁₈ alkylamido betaines, preferably alkylamido propyl betaines, based on the total weight of the composition comprising alkylamido betaines.

In a preferred embodiment, the composition comprising alkylamido betaines comprises 0.2 to 4 wt.-% of C₆ alkylamido betaines, preferably alkylamido propyl betaines, 3 to 7 wt.-% of C₈ alkylamido betaines, preferably alkylamido propyl betaines, 2 to 6 wt.-% of C₁₀ alkylamido betaines, preferably alkylamido propyl betaines, 35 to 45 wt.-% of C₁₂ alkylamido betaines, preferably alkylamido propyl betaines, 5 to 13 wt.-% of C₁₄ alkylamido betaines, preferably alkylamido propyl betaines, and 4 to 10 wt.-% of C₁₆ alkylamido betaines, preferably alkylamido propyl betaines, each based on the total weight of the composition comprising alkylamido betaines.

In a preferred embodiment, the composition comprising alkylamido betaines comprises at least 90 wt.-% of C₆ alkylamido betaines, preferably alkylamido propyl betaines, based on the total weight of the composition comprising alkylamido betaines.

In a preferred embodiment, the composition comprising alkylamido betaines comprises at least 95 wt.-%, based on the total weight of the composition comprising alkylamido betaines, of C₁₂-14 alkylamido betaines, preferably alkylamido propyl betaines, and further comprises 36 to 46 wt.-%, preferably 38 to 42 wt.-%, of a C₁₂ alkylamido betaines, preferably alkylamido propyl betaine, and 6 to 13 wt.-%, preferably 8 to 11 wt.-%, of a C₁₄ alkylamido betaines, preferably alkylamido propyl betaine, each based on the total weight of the composition comprising alkylamido betaines.

The present invention further relates to a composition comprising alkylamido betaines obtained by the process outlined in the following.

Generally, betaines can be prepared by reacting a carboxylic acid with a di-functional amine and subsequently reacting the resulting product with a quaternizing agent, preferably a chloroacetic acid. Methods for preparation of such compounds can be found for example in DE2926479A, DE4340423C₁, DE4207386C₁ and DE4430084A1 respectively. Generally, the reaction can start directly from the oil source, i.e., by amidation of the glycerides of the Macaúba palm oil, or from a fatty acid composition derived from said oil, i.e., by amidation of the free fatty acids.

In case the betaines are prepared from a fatty acid composition, the preparation method further comprises a preceding conversion step of preparing the fatty acid composition from Macaúba palm oil. In a preferred embodiment, the conversion step is conducted under chemical or enzymatic conditions, preferably under chemical conditions.

In a preferred embodiment, the conversion step involves a hydrolysis. Any suitable hydrolysis method can be conducted. In a preferred embodiment, the hydrolysis is preferably performed under chemical conditions, preferably in the presence of a catalyst. In another embodiment, the hydrolysis is preferably performed under enzymatic conditions, preferably at a temperature of 32 to 40° C.

The conversion step may further comprise partial or complete hydrogenation, wherein the double bonds of the fatty acid moieties are completely or partially removed. If the conversion step comprises a complete hydrogenation, the fatty acid composition does not comprise unsaturated moieties.

As a side product of the hydrolysis glycerol can be provided, preferably after a refine step. In a preferred embodiment, the conversion step further comprises the step of separating off glycerol.

Hence, the process for the preparation of the composition comprising amidoalkyl betaines of the present invention a process comprises the steps of:
a) subjecting Macaúba palm oil or a fatty acid composition derived from Macaúba palm oil to a reaction with 3-dimethylaminopropyl amine yielding an intermediate; and
b) subjecting the intermediate to a reaction with chloroacetic acid or sodium mono-chloroacetate yielding the alkylamido betaines.

Hence, the preparation is conducted preferably in a two-step process: in the first step the Macaúba palm oil or a fatty acid composition derived therefrom is reacted with dimethylaminopropyl amine yielding an amidoamine. i.e., the intermediate. The reaction takes place at a temperature in the range of from 140° C. to 210° C., and needs two to six hours, preferably four hours. Preferably, the resulting amidoamine is washed to eliminate any excess of remaining amine or distilled under vacuum, preferably at a temperature in the range of 130 to 170° C.

In the second step the resulting amidoamine reacts with a quaternizing compound, preferably cloroacetic acid or sodium mono-chloroacetate, more preferably sodium mono-chloroacetate. The reaction can be understood as a quaternization reaction of a tertiary amine with a monochloroacetate as an alkylating agent. Preferably, a high degree of conversion can be achieved using monochloroacetate. It is believed that this is preferred, because the free acid could slightly hinder the amine function.

Therefore, preferably, weakly alkaline conditions, analogous to the dissociation degree of monochloroacetic acid, are preferred. Also preferably, a slight excess of monochloroacetate is used to increase the yield. The reaction is preferably carried out in an aqueous solution. Preferably, the amide of the first step is added at a temperature in the range of 70 to 90° C., preferably 80-85° C. Subsequently, the temperature is kept for 2 to 6 hours, preferably 3 hours.

During the reaction, it is preferred to add after each hour the corresponding amount of NaOH to keep the pH value of the solution. This limits the number and amounts of undesired by-products. The pH value is preferably maintained at a value between 8 and 9 during the reaction.

Hence, the present invention relates to a composition comprising alkylamido betaines obtainable by a process as set out above.

The composition comprising alkylamido betaines obtainable by a process as set out above are aqueous compositions, preferably having a water content of 30-60 wt % and a pH of about 4.5-6.5.

Preferred embodiments are already above-outlined for the composition comprising alkylamido betaines derived from Macaúba palm oil or Macaúba pulp oil and shall apply for the composition comprising alkylamido betaines obtainable by a process as set out above, as well.

In a preferred embodiment of the present invention, composition comprising alkylamido betaines obtainable by a process as set out above can be further dried to powders, having a water content of 10 wt % or less, preferably 5 wt % or less.

As indicated above, the present invention further relates to the use of the above-outlined compositions as an amphoteric surfactant in a personal care composition or a cleaning composition.

In a preferred embodiment, the above-outlined compositions are used as an amphoteric surfactant in a personal care composition, preferably selected from the group consisting of face care composition, hair care composition, body care composition, oral care composition and baby care compositions.

Suitable cosmetic formulations containing active ingredients are, e.g., hormone preparations, vitamin preparations, vegetable extract preparations and antibacterial preparations.

According to the present invention the personal care composition may comprise one or more active agent(s), e.g., organic and inorganic UV filters and vitamins, as well as other ingredients or additives, e.g., pigments, emulsifiers, emollients, lipid layer enhancers, moisturizers, viscosity regulators, stabilizers, preservatives, pH agents, conditioning polymers, pearlizers, opacifiers or fragrances.

In a preferred embodiment, the above-outlined compositions are used as amphoteric surfactants in a sunscreen.

In a preferred embodiment, the above-outlined compositions are used as amphoteric surfactants in a decorative preparations, preferably selected from the group consisting of lipsticks, nail varnishes, eye shadows, mascaras, dry and moist make-up, rouge, powders, depilatory agents and suntan lotions.

The personal care composition is preferably in form of creams, ointments, pastes, foams, gels, lotions, powders, make-ups, micellar waters, sprays, sticks, bars, tablets, or aerosols.

Preferably the personal care compositions are aqueous compositions, preferably having a water content of 70-95 wt % and a pH of 4.0-8.0, more preferably a pH of 4.5-6.0. However, alternatively, personal care compositions can also be processed to powders by drying. Such powders preferably have a water content of less than 10 wt %, more preferably less than 5 wt %.

Preferably, the amphoteric surfactant is used to improve the viscosity, improve the mildness, improve dispersing, emulsifying and/or solubilization, improve the stability, appearance and/or feel and/or improve the foaming behavior.

In a preferred embodiment, the above-outlined compositions are used as amphoteric surfactants in a cleaning composition, preferably selected from the group consisting of home care formulation, industrial care formulation, and institutional care formulation.

In a preferred embodiment, the cleaning composition is selected from the group consisting of laundry composition and/or detergents (personal and commercial), dishwashing composition (manual and automatic), hard surface cleaning composition, food service and kitchen hygiene composition, food and beverage processing composition, sanitation composition, institutional cleaning composition, industrial cleaning composition, and vehicle and transportation care composition.

The cleaning composition may comprise at least one bleaching system known in the art in an amount of from up to 50 wt.-%. Suitable bleaching components include bleaching catalysts, photobleaches, bleach activators, sources of hydrogen peroxide such as sodium percarbonate and sodium perborates, preformed peracids, and mixtures thereof.

The cleaning compositions may furthermore comprise dirt-suspending agents, for example sodium carboxymethylcellulose; pH regulators, for example alkali metal or alkaline earth metal silicates; bactericides; foam regulators, for example soap; salts for adjusting the spray drying and the granulating properties, for example sodium sulfate; fragrances; antistatic agents; fabric conditioners; further bleaching agents; pigments; and/or toning agents.

Preferably, the amphoteric surfactant is used to control the metal ions, improve the dispersing, improve the emulsifying, improve the mildness, improve material compatibility, control the foaming, modify the surface, and/or protect the ingredient(s).

In a preferred embodiment, the personal care composition or the cleaning composition comprises at least two surfactants. In this connection it is to be understood that the personal care composition or the cleaning composition may comprise at least two above-outlined surfactants, at least three of the above-outlined surfactants or at least one of the above-outlined surfactant in combination with at least one further, different surfactant. The at least one further, different surfactant may be an amphoteric surfactant.

As indicated above, the present invention further relates to a personal care composition or a cleaning composition comprising a composition as above-outlined in more detail.

Preferred embodiments are already above-outlined for the use of above-outlined compositions as amphoteric surfactants in a personal care composition or a cleaning composition and shall apply for the personal care composition or a cleaning composition, as well.

### Experimental Part

The present invention is further illustrated by the following examples.

### Measurement Methods

### a) Viscosity

The viscosity of the compositions comprising betaines as well as the shampoo and body wash formulations has been measured using a Brookfield JAC-ML-LAB Rotational Viscosimeter according to ASTM D 2196.

### b) Color

The color of the compositions comprising betaines as well as the shampoo and soap formulations has been measured on a Lovibond Colorimeter using the Gardener Color Scale (ASTM D1544 and ASTM D1209) and the Lovibond RYBN Color Scale (AOCS Dd-5-92)

### c) Foaming Power

200 ml of a 1% solution of the surfactant in a 1000 ml beaker is stirred according to the conditions of this method (cf. below). The foam formed is evaluated by the volume immediately after stirring (time zero) and after 5 and 15 minutes standing time.

2 g of the sample are weighed into a 1000 ml beaker and dissolved in 200 ml of distilled water. The solution is stirred 30 times with a stirring rod, moving from top to bottom, through the space of the beaker. Thereafter, a the first reading is taken: the upper height of the foam (ml) is subtracted by the lower height of the foam (ml) (reading at time zero (0). This reading is repeated after 5 and 15 min waiting time.

### EXAMPLES

The following examples are considered for the Macaúba palm (e.g. having registration number AEB402A) having an oil yield in tons per hectare per year of about 9.0 t/ha/yr.

### Inventive Example IE1

The Macaúba palm is planted on a cattle field, e.g. about 380 trees per hectare. No deforestation is needed since the Macaúba palms are cultivated on already existing fields (silvopastoral) and the farmer can in addition to cattle breeding and/or milk production distribute the Macaúba fruits.

### Inventive Example IE2

The Macaúba palm is planted on soya plantation (having a growth height of about 20 to 80 cm and an oil yield in tons per hectare per year about 0.6 t/ha/yr), e.g. about 340 trees per hectare. Again, no deforestation is needed since the Macaúba palms are cultivated on an already existing plantation (agroforestry). As the Macaúba palm grows up to about 15 meters in height, the soya can be cultivated parallel. In this connection, it is also possible to cultivate at least one more additional different plant (having a growth height of about 1 to 7 m) such as sunflower (having an oil yield in tons per hectare per year of about 0.7 t/ha/yr) or beans parallel.

As can be seen from the above examples, deforestation can be significantly reduced by cultivating Macaúba palms. Further, the biodiversity can be increased. In addition, even if the Macaúba palm is not cultivated as a monoculture, the total oil yield can be comparable with an oil palm (having an oil yield in tons per hectare per year of about 3.8 t/ha/yr) monoculture since the oil yield as above defined of the Macaúba palm is higher. Without being bound to any theory, using a plant having an improved oil yield, degraded areas and springs and watersheds can more easily recover. Further, the retention of moisture in the soil is improved.

### Example CE1—Cocamidylbetain Preparation from Palm Kernel Oil (PKO)

The preparation is conducted preferably in a two-step process: in the first step the PKO is reacted with dimethylaminopropyl amine. The reaction takes place in a temperature range from 140° C. up to 210° C., and needs approximately four hours. The resulting amidoamine is washed to eliminate any excess of amine. In the second step the amidoamine of the PKO reacts with a quaternizing compound, preferably sodium mono-chloroacetate. The reaction can be understood as a quaternization reaction of a tertiary amine with a monochloroacetate as an alkylating agent. A high degree of conversion occurs if during the alkylation process a salt is formed with mono-chloroacetate, because the free acid would hinder the amine function. Therefore, weakly alkaline conditions, analogous to the dissociation degree of monochloroacetic acid, are recommended. Also, a slight excess of monochloroacetate increases the yield. The reaction is carried out in an aqueous solution, and the amidoamine of PKO from the first step is added at 80-85° C. Then the temperature is kept at 80-85° C. for three hours. During the reaction, it is preferred to add each hour the corresponding amount of NaOH. This limits the number and amount of undesired by-products. The pH during the reaction should be maintained at a value between 8 and 9. The final product is yielded in a concentration of 20% by weight up to at maximum 50% by weight of betaine. The sterol content of the final product is at maximum 1000 ppm.

### Inventive Example IE3—Cocamidylbetain Preparation from Macaúba Palm Oil (MAC)

The betaine is prepared according to the method as provided in CE1 with the exception that instead of PKO, MAC has been used.

### Comparative Example CE2—Stability of PKO Derived Betaines

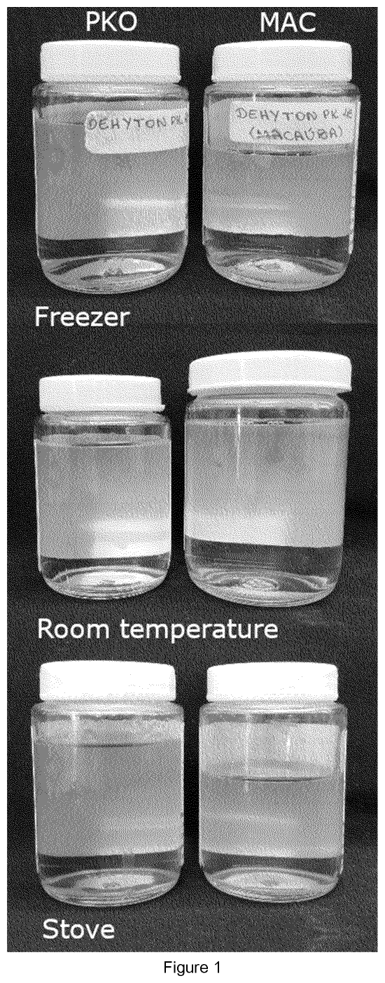

Three batches of the product prepared according to CE1 were stored for three months in a) a freezer at 5° C.; b) at room temperature (21° C.); and c) in a stove at 50° C. The storage of a), b), and c) ensured that no light could reach the batches during the stability test. After three months, the batches were taken out and their pH was measured as well as the color has been determined. Respective values can be found in Table 1 as well as a comparison of the colors in FIG. 1.

### Inventive Example IE4—Stability of MAC Derived Betaines

The method of CE2 has been used with the exception that instead of betaines derived from PKO, betaines derived from MAC have been used

### Comparative Example CE3—Viscosity, Foam, and Stability of a PKO Derived Shampoo

A shampoo is prepared according to the amounts as shown in Table 1 by homogenizing phase A. Subsequently, the ingredients of phase B are added to the homogenized phase A in sequence, whereby the resulting phase is homogenized in between each addition. Further subsequently, Kathon CG is added, and the phase homogenized. Finally, sodium chloride is slowly added thereby observing an increase of the viscosity of the phase. Finally, the pH value is corrected to 5.0-6.5.

### Inventive Example IE5—Viscosity, Foam, and Stability of a MAC Derived Shampoo

The method of CE3 has been used with the exception that instead of betaines derived from PKO, betaines derived from MAC have been used.

TABLE 1
Formulations of the shampoos of CE3 and IE5

| | | | CE3 | IE5 | |
|---|---|---|---|---|---|
| | Trade Name | INCI Name | % | % | Function |
| A | Water, demin. | Aqua | 56.96 | 56.96 | Solvent |
| | DEHYQUART ® GUAR HP | Guar Hydroxypropyl- | 0.20 | 0.20 | Conditioning |
| | | trimonium Chloride | | | Polymer |
| B | CE1 | Cocamidopropyl Betaine | 7.97 | — | Amphoteric |
| | | | | | Surfactant |
| | IE3 | Cocamidopropyl Betaine | — | 7.97 | Amphoteric |
| | | | | | Surfactant |
| | TEXAPON ® HBN DM | Sodium Laureth Sulfate | 32.40 | 32.40 | Anionic |
| | | | | | Surfactant |
| | LAMESOFT ® PO 65 | Coco-Glucoside (and) | 0.50 | 0.50 | Lipid Layer |
| | | Glyceryl Oleate | | | Enhancer |
| | PLANTACARE ® 818 UP | Coco-Glucoside | 1.00 | 1.00 | Nonionic |
| | | | | | Surfactant |
| C | KATHON CG | CMIT/MIT | 0.02 | 0.02 | Preservative |
| D | Sodium Chloride | Sodium Chloride | 0.95 | 0.95 | Thickener |

### Comparative Example CE4—Viscosity, Foam, and Stability of a PKO Derived Body Wash

A body wash is prepared according to the amounts a shown in Table 2 by adding Texapon® HBN and the product of example 3 in water, in sequence, and homogenizing with each addition. Subsequently, Kathon CG is added, and the phase homogenized. Finally, sodium chloride is added slowly thereby increasing the viscosity. The pH value is corrected to 5.0-6.5. Viscosity, foam development and stability of the resulting product has been measured.

### Inventive Example IE6—Viscosity, Foam, and Stability of a PKO Derived Body Wash

A body wash is prepared according to the amounts a shown in Table 2 by adding Texapon® HBN and the product of example 3 in water, in sequence, and homogenizing with each addition. Subsequently, Kathon CG is added, and the phase homogenized. Finally, sodium chloride is added slowly thereby increasing the viscosity. The pH value is corrected to 5.0-6.5.

TABLE 2
Formulations of the body wash of CE4 and IE6

| | | | CE4 | IE6 | |
|---|---|---|---|---|---|
| | Trade Name | INCI Name | % | % | Function |
| A | TEXAPON ® HBN DM | Sodium Laureth Sulfate | 30.00 | 30.00 | Anionic |
| | | | | | Surfactant |
| | CE1 | Cocamidopropyl Betaine | 4.74 | — | Amphoteric |
| | | | | | Surfactant |
| | IE3 | Cocamidopropyl Betaine | — | 4.74 | Amphoteric |
| | | | | | Surfactant |
| B | Water, demin. | Aqua | 32.40 | 32.40 | Solvent |
| C | Kathon CG | CMIT/MIT | 0.02 | 0.02 | Preservative |
| D | Sodium Chloride | Sodium Chloride | 2.35 | 2.35 | Thickener |

### Discussion of the Results

The comparison of the data retrieved in CE2 and IE4 are summarized in table 3. From these data it can be seen that in case of betaines derived from Macaúba palm oil (IE4), the pH value after storage over 3 months stays constant independently of the temperature used during storage in comparison to betaines derived from Palm Kernel Oil. Furthermore, as shown by the pictures showing colors in FIG. 1, it can be seen that the betaines derived from Macaúba palm oil tend less to colorization during storage. Thus, in summary, betaines derived from Macaúba palm oil show better stability during storage and in particular shows temperature independent stability during storage in comparison to betaines derived from Palm Kernel Oil.

TABLE 3
Comparison of pH and color results of CE2 and IE4

| | | CE2 | IE4 |
|---|---|---|---|
| Initial | pH | 4.5-5.5 | 4.5-5.5 |
| Storage for 3 months | pH | 5.21 | 5.35 |
| at 5° C. (Freezer) | | | |
| Storage for 3 months | pH | 5.12 | 5.35 |
| at Room temperature | | | |
| Storage for 3 months | pH | 4.72 | 5.35 |
| at 50° C. (Stove) | | | |

The comparison of the data retrieved in CE3, CE4, IE5, and IE6 are summarized in table 4. From these data it can be concluded that the viscosity of a shampoo and/or a body wash derived from Macaúba palm oil is on a comparable level in comparison to the viscosity derived from Palm Kernel Oil. It can be further seen that this property is achieved at acceptably constant stability of the composition during storage. Finally, the shampoo and soaps derived from Macaúba palm oil show improved foam development.

TABLE 4
Results of the measurements of viscosity, pH, and stability

| | | CE3 | IE5 | CE4 | IE6 |
|---|---|---|---|---|---|
| Viscosity (initial) | [cP] | 4280 | 3680 | 6400 | 5740 |
| Viscosity (3 months/5° C.) | [cP] | 3860 | 3440 | 5320 | 4900 |
| Viscosity (3 months/25° C.) | [cP] | 3880 | 3340 | 5460 | 5200 |
| Viscosity (3 months/45° C.) | [cP] | 6560 | 5400 | 7600 | 7760 |
| pH (initial) | | 5.53 | 5.46 | 5.42 | 5.47 |
| pH (3 months/5° C.) | | 5.62 | 5.54 | 5.77 | 5.84 |
| pH (3 months/25° C.) | | 5.60 | 5.52 | 5.74 | 5.78 |
| pH (3 months/45° C.) | | 5.65 | 5.41 | 5.89 | 5.79 |
| Foam (0 min) | [ml] | 850 | 945 | 830 | 845 |
| Foam (5 min) | [ml] | 740 | 810 | 710 | 765 |
| Foam (15 min) | [ml] | 650 | 720 | 640 | 675 |

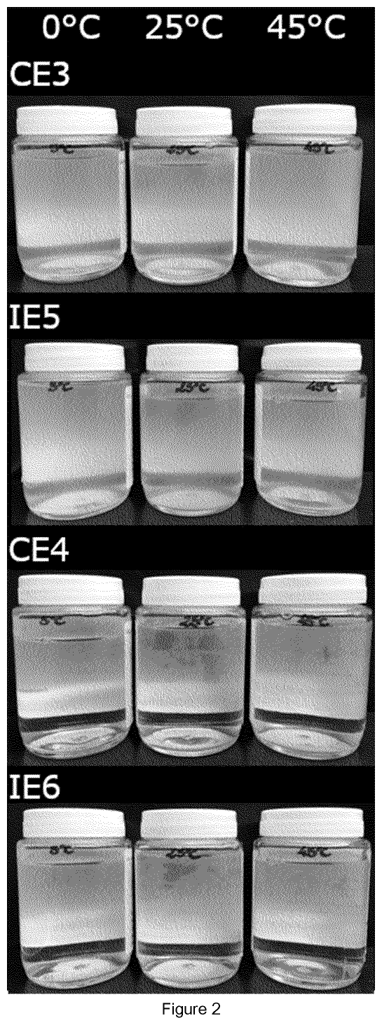

The colors of the resulting stored shampoos and body washs have been depicted in FIG. 2. In parallel to the findings for the pure betaine compositions, also the shampoos and body washs derived from PKO show a higher yellow color intensity compared to their MAC counterparts, further proving the increased storage stability of shampoo and body wash compositions comprising MKO based betaines.

1. A composition comprising alkylamido betaines, wherein the alkylamido betaines are derived from Macaúba palm oil.

2. The composition according to claim 1, wherein the Macaúba palm oil is obtained by extraction of the fruits, palm pulp, and/or palm kernel of the Macaúba palm.

3. The composition according to claim 1, comprising at least 45 wt.-%, based on the total weight of the composition, of C₄-C₂₂ alkylamido betaines.

4. The composition according to claim 1, wherein the alkylamido betaines are a mixture of compounds of the general formula (I)
R¹—CONH—X—NR²R³—R⁴—Y (I)
wherein R¹ is an alkyl or alkenyl moiety containing 4 to 22 carbon atoms, X is a (CH₂)ₙ group, wherein n is an integer from 1 to 6, R² and R³ independently are an alkyl moiety with 1 to 4 carbon atoms or an hydrogen atom, R⁴ is a C₁-alkylene, and Y is a COO⁻ group.

5. The composition according to claim 4, wherein the mixture of compounds of formula (I) R² is identical to R³.

6. The composition according to claim 4, wherein X in formula (I) is a (CH₂)₃-group.

7. The composition according to claim 1, wherein the alkylamido betaines are cocamido betaines.

8. The composition according to claim 1, wherein the composition comprises:
1 to 20 wt.-% of a C₈ alkylamido betaine,
1 to 8 wt.-% of a C₁₀ alkylamido betaine,
30 to 48 wt.-% of a C₁₂ alkylamido betaine,
5 to 15 wt.-% of a C₁₄ alkylamido betaine,
4 to 13 wt.-% of a C₁₆ alkylamido betaine,
15 to 42 wt.-% of a C₁₈ alkylamido betaine, and
0 to 5 wt.-% of a C₂₀ alkylamido betaine,
each based on the total weight of the composition.

9. The composition according to claim 1, wherein the composition comprises:
3 to 7 wt.-%, of a C₈ alkylamido betaine,
2 to 6 wt.-%, of a C₁₀ alkylamido betaine,
36 to 46 wt.-%, of a C₁₂ alkylamido betaine,
6 to 13 wt.-%, of a C₁₄ alkylamido betaine,
5 to 11 wt.-%, of a C₁₆ alkylamido betaine,
25 to 40 wt.-%, of a C₁₈ alkylamido betaine, and
0 to 4 wt.-%, of a C₂₀ alkylamido betaine,
each based on the total weight of the composition.

10. A composition comprising alkylamido betaines, obtainable by a process comprising the steps of:
a) subjecting Macaúba palm oil or a fatty acid composition derived from Macaúba palm oil to a reaction with 3-dimethylaminopropyl amine yielding an intermediate; and
b) subjecting the intermediate to a reaction with chloroacetic acid or sodium mono-chloroacetate yielding the alkylamido betaines.

11. (canceled)

12. A personal care composition or a cleaning composition comprising a composition according to any of claim 1.

13. (canceled)

14. (canceled)
